SEQUENCE LISTING (20 sequences)

SEQ ID NO: 1 - DNA, 29 bp, Homo sapiens
gagctccgttttcctctgcagagacttcg

SEQ ID NO: 2 - DNA, 30 bp, Homo sapiens
gccccggcgcaacggtggcatgcctctctg

SEQ ID NO: 3 - DNA, 30 bp, Homo sapiens
gccccggcgccacggtggcatgcctctctc

SEQ ID NO: 4 - DNA, 30 bp, Homo sapiens
atctctccgcgtgactcttgccaccgccac

SEQ ID NO: 5 - DNA, 30 bp, Homo sapiens; identical to SEQ ID NO: 4

SEQ ID NO: 6 - DNA, 30 bp, Homo sapiens
gcagccgtgcatctctccgcgtgactcttg

SEQ ID NO: 7 - DNA, 30 bp, Homo sapiens
gtgcgcttatgcagctgtgcacctgcagta

SEQ ID NO: 8 - DNA, 30 bp, Homo sapiens
gagcacacacccggtggacaccgtaaaggc

SEQ ID NO: 9 - DNA, 30 bp, Homo sapiens
gagttgcgttctctgtagcacagaccctaa

SEQ ID NO: 10 - DNA, 28 bp, Homo sapiens
gcccctgtgaatcctgtacaccgagatg

SEQ ID NO: 11 - DNA, 30 bp, Homo sapiens
gcagccctcaacaatcagggtcagagtcca

SEQ ID NO: 12 - DNA, 27 bp, Homo sapiens
gactcgcagagcatggcgagggcgcag

SEQ ID NO: 13 - DNA, 30 bp, Homo sapiens
ggcgctgcgcctttgcgaaagtgaagctgt

SEQ ID NO: 14 - DNA, 38 bp, Homo sapiens
gcctttgcgaaagtgaagctgtgttctcatcagcagag

SEQ ID NO: 15 - DNA, 30 bp, Homo sapiens
gccacccgccaatacatttggagcaagagg

SEQ ID NO: 16 - DNA, 33 bp, Homo sapiens
gctttgacacgactcggggcttcattgatggtg

SEQ ID NO: 17 - DNA, 33 bp, Homo sapiens
gctttggcacgactcggggcttcattgatggtg

SEQ ID NO: 18 - DNA, 33 bp, Homo sapiens
gcctttgcgagggcggagttgcgttctctttag

SEQ ID NO: 19 - DNA, 29 bp, Homo sapiens
gccgtgcgactttgctcctgcaacacacg

SEQ ID NO: 20 - DNA, 36 bp, Homo sapiens
gggacaaggcaattgtcatgcaattatctatgcagg

(19) United States
(12) Patent Application Publication
(10) Pub. No.: US 2014/0024034 A1
(43) Pub. Date: Jan. 23, 2014

(54) NOVEL PRIMERS FOR DETECTING HUMAN CHROMOSOME END-TO-END TELEMORE FUSION

(71) Applicant: Indiana University Research and Technology Corporation, Indianapolis, IN (US)

(72) Inventor: Hiromi Tanaka, Indianapolis, IN (US)

(21) Appl. No.: 13/934,266

(22) Filed: Jul. 3, 2013

Related U.S. Application Data
(60) Provisional application No. 61/672,549, filed on Jul. 17, 2012.

(51) Int. Cl. C12Q 1/68

(52) U.S. Cl. CPC C12Q 1/6883
USPC 435/6.12; 536/24.33

(57) ABSTRACT

The present disclosure is directed to compositions and methods for detecting signs of telomere dysfunction as diagnostic indicators of metastatic disease. More particularly, diagnostic reagents and procedures are provided for analyzing samples to detect the presence of telomere fusions as an early diagnostic test for cancerous or pre-cancerous cells.

### CROSS REFERENCE TO RELATED APPLICATIONS

This application claims priority to U.S. Provisional Patent Application No. 61/672,549 filed on Jul. 17, 2012, the disclosure of which is hereby expressly incorporated by reference in its entirety.

### FIELD OF THE INVENTION

The invention relates generally to medicine, and more particularly to detecting human chromosome end-to-end telomere fusion in cancer.

### BACKGROUND

Genomic instability is one of the earliest neoplastic changes known to occur in tumorigenesis. Several recent reports indicate that defects in telomere maintenance may play an important role in the development of cancer, and more particularly, breast cancer. Telomeres are specialized DNA/protein structures, functioning as protective caps to prevent chromosome end fusions and permit DNA end replication. In healthy cells, telomere length is highly regulated in a tissue and cell-type specific manner and is dependent on mitotic turnover rate, telomerase activity and telomerase-associated factors. As cells divide, the length of the telomeres shrinks until the telomere reaches a critical size wherein the cell stops dividing.

Telomerase is a ribonucleoprotein complex consisting of a reverse transcriptase catalytic subunit (hTERT) and an RNA (hTER) that supplies the template for (T₂AG₃) repeat addition, among other essential functions. Telomerase is absent, or greatly reduced, in most somatic cells resulting in progressive telomere shortening after each cell cycle that can lead to loss of telomere function. Telomerase is activated early in the progression of breast cancer, and is present in ductal carcinoma in situ (DCIS). Remarkably, telomerase is activated in over 95% of tumors via a complex process, including loss of hTERT-specific negative transcription regulators, likely caused by earlier events of genomic instability and hTERT-specific positive transcription activators. Therefore, hTERT activation is the most frequent gene regulatory alteration known to occur in most cancers and potentially an extremely useful marker. However, there are likely earlier events that are also associated with tumorigenesis that may provide for earlier detection of cancerous or pre-cancerous cells.

Several lines of evidence from mouse and human systems have suggested that defects in telomere maintenance play an important role in the development of cancer. Induction of telomere dysfunction by deficiency in the telomerase RNA component (mTER) in a p53 mutant mouse background results in significant levels of breast adenocarcinomas and colon carcinomas. Telomere dysfunction has also been shown to occur in a human mammary epithelial cell (HMEC) culture model. In this model, late passage HMECs escape a stress-associated senescence-like barrier and acquire genomic alterations including telomere fusions. In addition, clinical studies have shown that a significant proportion of normal breast luminal cells and ductal carcinoma in situ (DCIS) tissues have shortened telomeric DNA lengths when assayed by telomere fluorescence in situ hybridization. Several studies have reported that anaphase bridges, possibly formed as a consequence of telomere dysfunction, are present in early staged tumors, including DCIS. However, while the presence of anaphase bridges and shortened telomeres may delineate a significant population of cells at risk for telomere dysfunction, it does not provide conclusive evidence of telomere dysfunction in human cancers. By contrast, direct evidence of telomere dysfunction in the form of high levels of telomere fusions was recently reported in the blood-borne cancer chronic lymphocytic leukemia (CLL) (Lin T T, et al. (2010) Blood 116:1899-1907).

To directly test whether telomere fusions are present in human early and later stage breast tumors, applicants have developed a highly specific and sensitive PCR-based technique referred to as TAR (telomere associated repeat) Fusion PCR. This technique allows for the detection and analysis of telomere dysfunction within human solid tumor tissue. Applicants have discovered that telomere fusions are present during the early DCIS stage and remained at sustained levels during invasive ductal carcinoma (IDC). The presence of telomere end-to-end fusions is a fundamental indication of and a marker for loss of telomere function. Accordingly, applicants anticipate that TAR Fusion PCR could have significant applications both in clinical early detection strategies and in increasing the understanding of basic mechanisms of telomere driven genomic instability in human cancers.

### BRIEF SUMMARY

The successful amplification of individual telomere fusion junctions in the human genome requires the construction of suitable PCR primers that are capable of specific binding to their target telomere. Chromosome ends consist of very repetitive elements and there are evolutionally analogous interstitial sequences in the genome thus complicating the construction of such primers. In accordance with one embodiment, TAR1 (Telomere associated repeat 1)-like sequences are used as PCR primers. TAR1 sequences were originally described by Brown, et al. (Cell, (1990) 63:119-132). The canonical TAR1 sequence are present within ˜2 kb of the beginning of nearly all sequenced (TTAGGG)ₙ-adjacent DNA. Known TAR1s share regions which are highly similar, although the part and degree of similarity can vary substantially. In one aspect of the present disclosure, a multiplexing assay is used to detect chromosome fusions, wherein several telomere fusion events can be co-amplified by using multiple primer pairs designed to hybridize to unique sequences within the TAR1 elements. Multiplex PCR with these telomere adjacent sequences is an advanced technique for detection of the chromosomal end-to-end fusion by amplifying multiple targets in a single PCR experiment.

Accordingly, the present disclosure is directed to diagnostic reagents and procedures for the early detection of cancer. More particularly, the present disclosure is directed to methods for analyzing samples to assess the existence of cancerous or pre-cancerous cells. In one embodiment the methods of the present disclosure are used to diagnose the existence of, or assess the risk of, breast, ovarian, prostate and colon cancer.

In accordance with one embodiment, a method of detecting telomere fusions in a biological sample as a diagnostic indicator of the existence of cancerous or pre-cancerous cells is provided. The method comprises contacting cellular DNA of a biological sample with two or more telomere specific PCR primers selected from the group consisting of SEQ ID NO: 1-20.

Suitable PCR reagents are combined with primers and the cellular DNA of a biological sample, and PCR is conducted. The production of an amplicon indicates the presence of one or more telomere fusions and is diagnostic for the presence of cancerous or precancerous cells in the biological sample.

In accordance with one embodiment a kit for screening biological samples for the presence telomere fusions is provided. In one embodiment the kit comprises, four or more PCR primers selected from the group consisting of SEQ ID. NO. 1-20

The kit can be further provided with one or more reagents for conducting PCR reactions or for detecting the amplified products produced by the PCR reaction.

The biological sample may be purified DNA from a patient's cells or the biological sample may be thin slices of cells or a tissue sample obtained from a patient. After conducting the PCR amplification the sample is screened for the presence of amplified products. Detection of an amplified product indicates the presence of telomere fusions and thus is diagnostic for the presence of cancerous or pre-cancerous cells in the patient's tissue.

These and other features, objects and advantages of the present disclosure will become better understood from the description that follows. In the description, reference is made to the accompanying drawings, which form a part hereof and in which there is shown by way of illustration, not limitation, embodiments of the invention.

These and other features, objects and advantages of the present invention will become better understood from the description that follows. The description of exemplary embodiments is not intended to limit the invention to the particular forms disclosed, but on the contrary, the intention is to cover all modifications, equivalents and alternatives falling within the spirit and scope of the invention as defined by the embodiments above and the claims below. Reference should therefore be made to the embodiments above and claims below for interpreting the scope of the invention.

### BRIEF DESCRIPTION OF THE DRAWINGS

The features, objects and advantages other than those set forth above will become more readily apparent when consideration is given to the detailed description below. Such detailed description makes reference to the following drawings, wherein:

FIGS. 1A & 1B are schematic drawings of the strategy used for the detection of telomere fusions using TAR Fusion PCR. FIG. 1A demonstrates that fusion junctions were amplified by multiplex primer sets (e.g., primer A, primer B) annealing within TAR1 regions adjacent to telomeric DNA sequences. The fusion junctions were detected by Southern analysis using a telomeric DNA [TTAGGG]4 probe. FIG. 1B demonstrates the chromosome locations of multiplex PCR primers used for TAR Fusion PCR. Black arrows represent primers and the distances (bp) from telomeric DNA starting positions. Open-ended arrows denote telomeric DNA. Open rectangle boxes denote genomic regions currently not available in current human genome sequence databases.

FIGS. 2A-2F. Telomere fusion analysis in cultured human male foreskin fibroblasts during telomere crisis. FIG. 2A is a photograph of chromosome spreads hybridized with a pancentromere probe to permit detection of dicentric chromosomes following telomere fusion. Chromosomes were stained with DAPI.

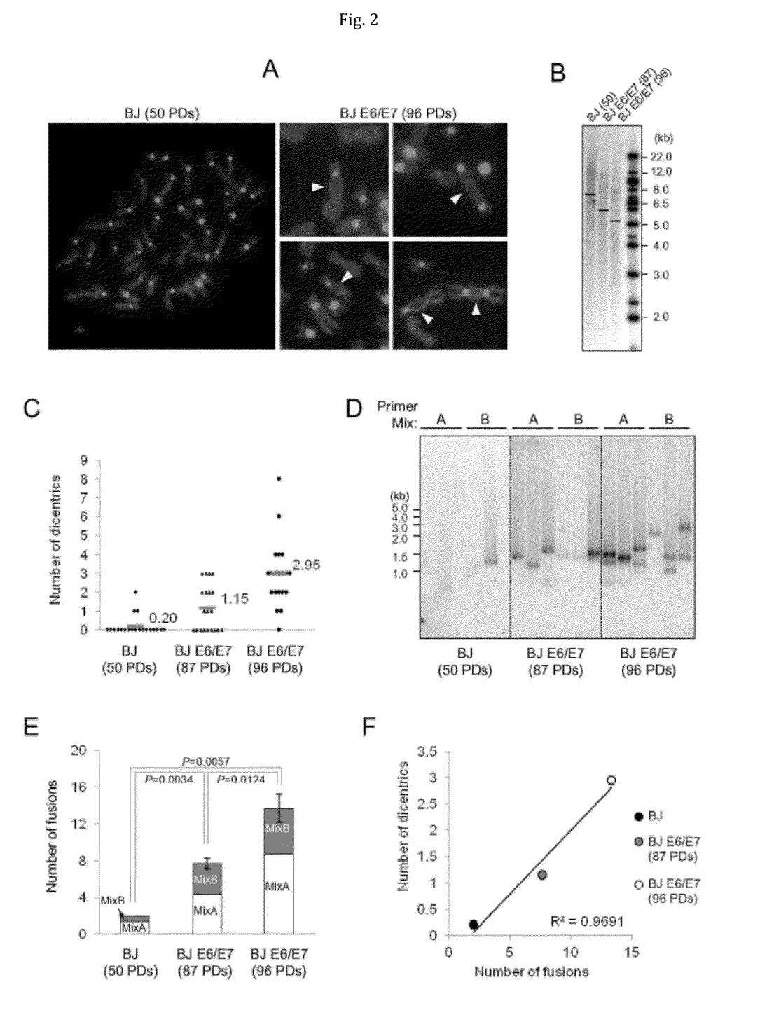

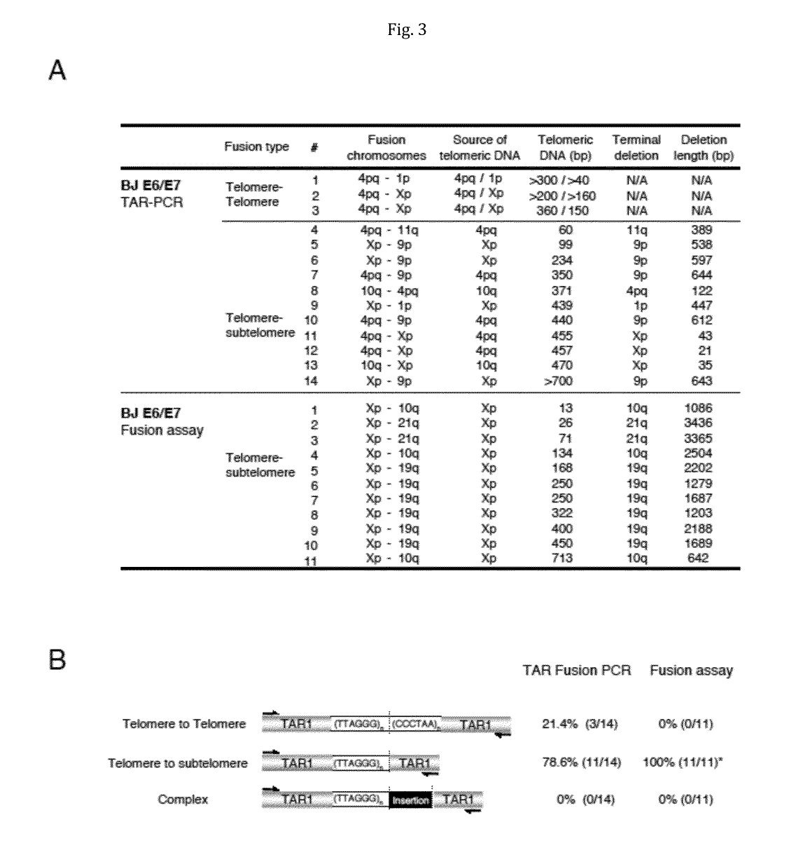

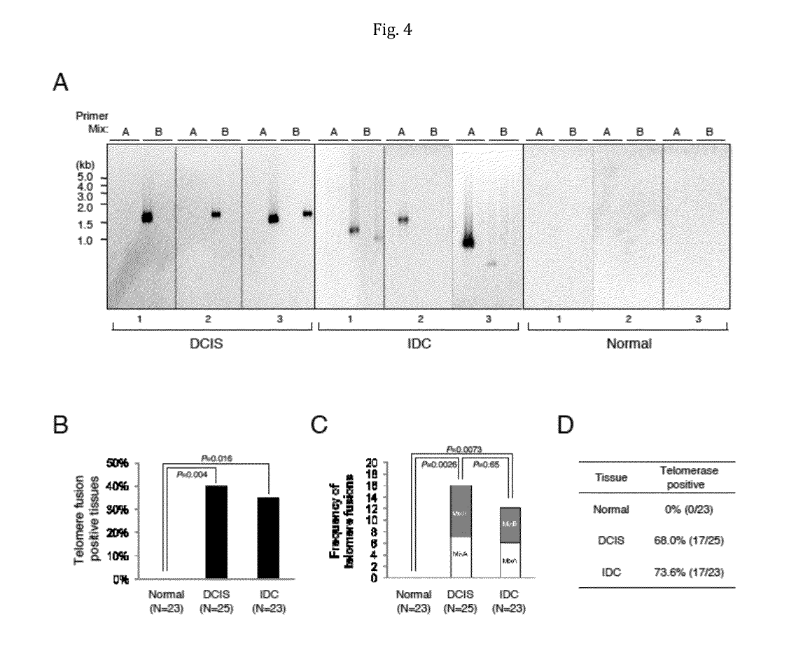

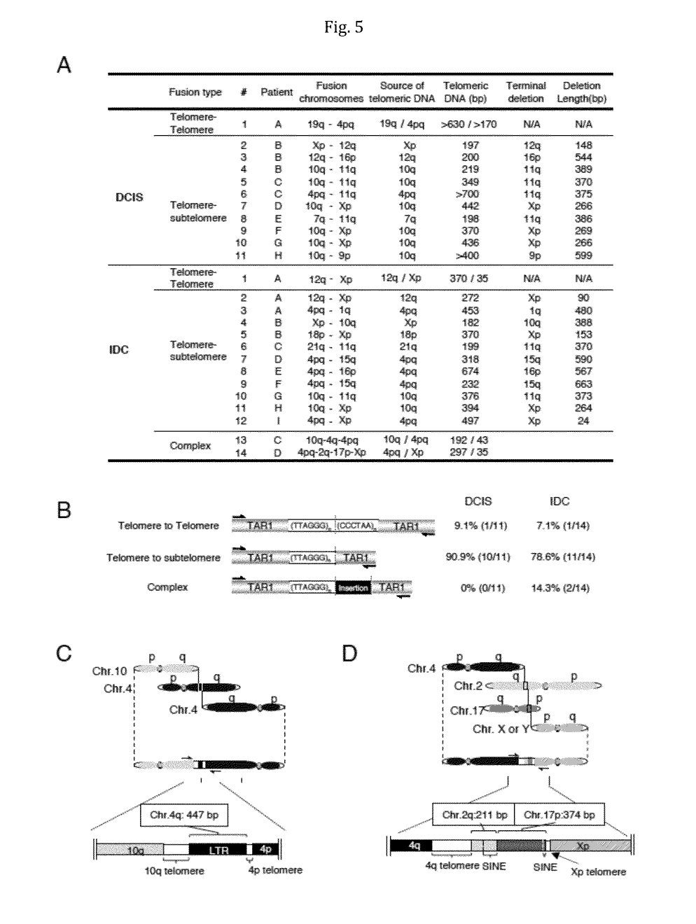

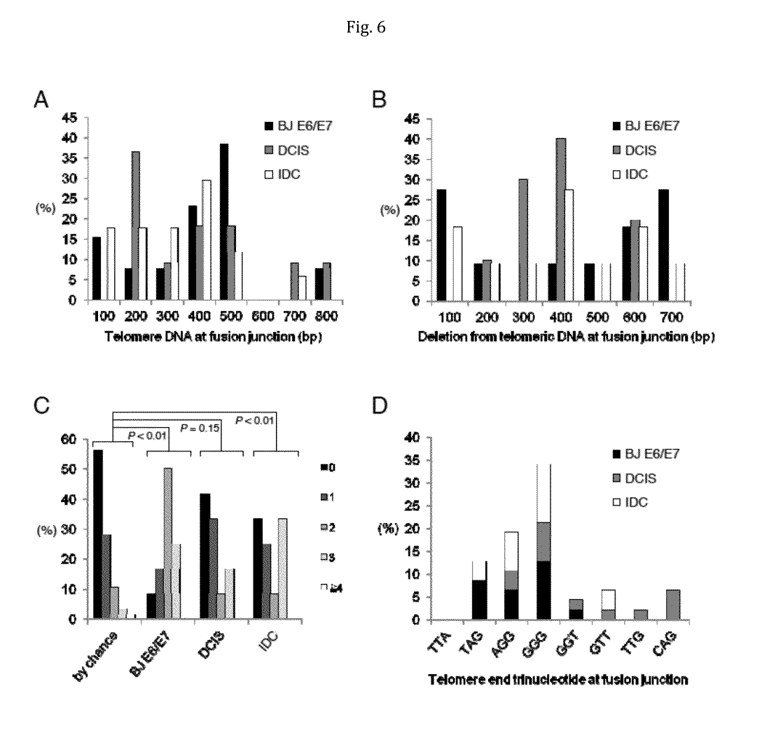

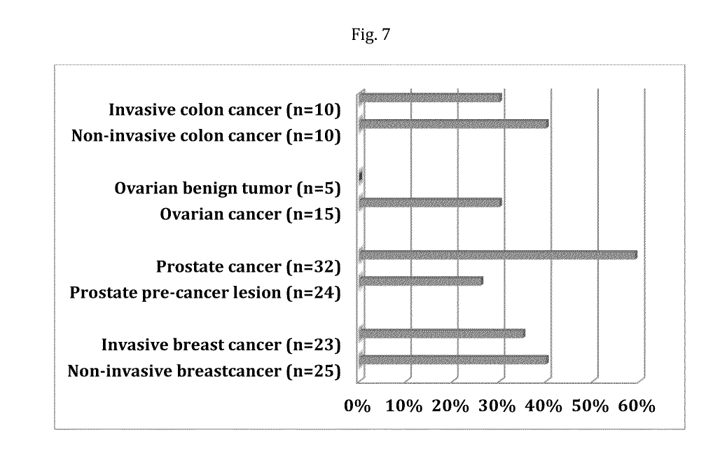

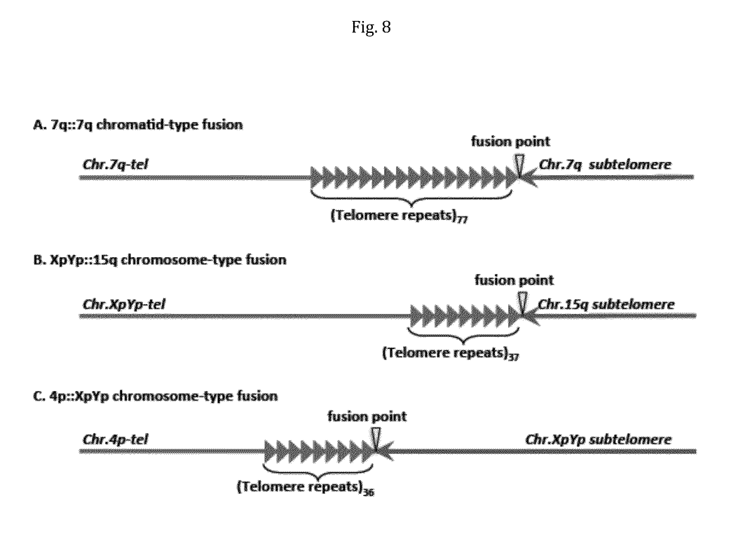

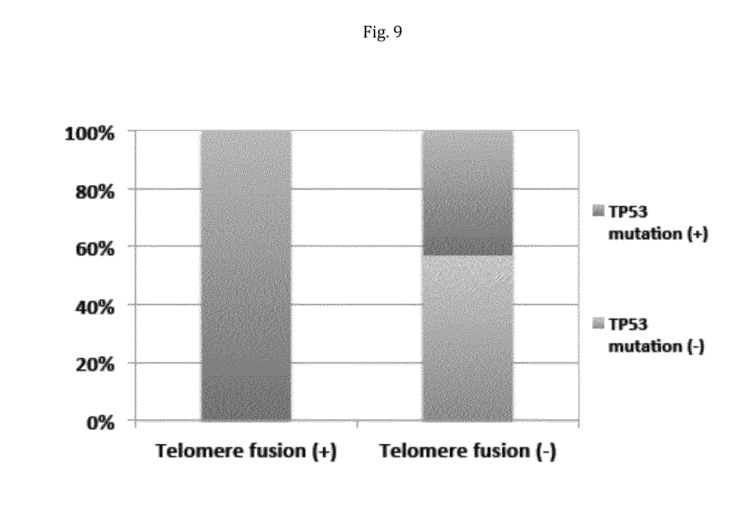

Left panel: a diploid BJ fibroblast metaphase spread at 50 population doublings (PDs). Right panel: example of a BJE6/E7 cell metaphase spread at 96 PDs exhibiting telomere fusions. Arrowheads indicate fusion junctions between two chromosomes. FIG. 2B is an autoradiograph showing the average frequency of dicentric chromosomes per metaphase. BJ cells at 50 PDs contained 0.20 dicentrics per metaphase; BJE6E7 cells at 87 PDs contained 1.15 per decentrics; BJE6E7 cells at 96 PDs contained 2.95 dicentrics chromosomes per metaphase. Twenty metaphase spreads were analyzed for each cell type at the referenced population doubling. FIG. 2C presents data from Southern blot analysis of mean telomere lengths. Mean telomere lengths are shown by a horizontal line upon each lane: BJ (50 population doubling (PD); 7.6 kb), BJ E6/E7 (87 PD; 5.9 kb) and BJ E6/E7 (96 PD; 5.2 kb). FIG. 2D is a Southern blot of TAR-fusion PCR products using a ³²P-labeled [TTAGGG]4 probe. Triplicate reactions are shown from multiplex primer mix A and B. FIG. 2E is a graph demonstrating the frequency of telomere fusions. The sum of the number of bands per six reactions is shown for each cell type from three independent TAR Fusion PCR experiments using the same DNA sample. FIG. 2F is a graph demonstrating the correlation between the metaphase telomere fusion analysis and TAR Fusion PCR. The y-axis represents the average number of dicentric chromosomes per metaphase as determined in FIG. 2C. The x-axis represents the average number of TAR Fusion PCR bands per six reactions as determined in FIG. 2E. White circles, BJ (50 PDs); gray circles, BJ E6/E7 (87 PDs); black circle, BJ E6/E7 (96 PDs). Note: the frequency of telomere fusion junctions using TAR Fusion PCR correlates with the frequency of dicentric chromosomes shown in panel B with R2=0.96914.

FIGS. 3A & 3B. Telomere fusion junction sequence analysis from BJ E6/E7 cells. FIG. 3A is a table presenting a summary of telomere fusion junction sequence analysis using TAR Fusion PCR and the Fusion Assay performed as previously described by Baird and colleagues (Letsolo et al (2010) Nucleic Acids Res 38:1841-1852). Fusion chromosomes=chromosomes involved in fusion junction. Source of telomeric DNA=chromosome presumed to contribute telomeric DNA within the fusion junction. Telomeric DNA (bp)=size of telomeric DNA at fusion junctions in bp. Terminal deletion=chromosome presumed to contain deletions within subtelomeric regions at fusion junctions. Deletion length (bp)=chromosomal deletion size of subtelomeric regions. N/A=not applicable. FIG. 2B provides a classification of telomere fusion types in BJ E6/E7 cells. Telomere-telomere fusion=fusion junctions containing telomeric DNA presumably from both chromosomal ends. Telomere-subtelomere fusion=junctions containing telomeric DNA presumably from one chromosome fused with another chromosome end with subtelomeric deletions. Complex fusion=fusion junctions containing non-telomeric DNA from interstitial chromosomal regions.

FIGS. 4A-4D. Telomere fusions in human breast tumor tissue. FIG. 4A presents Southern blot analysis of TAR-fusion PCR products using a ³²P-labeled [TTAGGG]4 probe. Six reactions from two primer sets A and B are shown for each patient sample. FIG. 4B is a graph demonstrating the percentage of human breast tumor tissues containing telomere fusions. N=total number of tissue samples analyzed. FIG. 4C is a graph showing the frequency of fusions within individual telomere fusion positive tumor tissue. The sum of the number of bands per six reactions is shown for each tissue positive for telomere fusions from three independent experiments. FIG. 4D is a table showing the percentage of human breast tissue positive for telomerase activity as assayed by the telomeric amplification protocol (TRAP).

FIGS. 5A-5D. Telomere fusion junction sequence analysis from breast tumor tissue. FIG. 5A is a table providing a summary of telomere fusion junction sequence analysis using TAR Fusion PCR. Fusion chromosomes=chromosomes involved in fusion junction. Source of telomeric DNA=chromosome presumed to contribute telomeric DNA within the fusion junction. Telomeric DNA (bp)=size of telomeric DNA at fusion junctions in basepairs. Terminal deletion=chromosome presumed to contain deletions within subtelomeric regions at fusion junctions. Deletion length (bp)=chromosomal deletion size of subtelomeric regions. N/A=not applicable. FIG. 5B provides a classification of telomere fusion types in DCIS and invasive tumors. Telomere-telomere fusion=fusion junctions containing telomeric DNA presumably from both chromosomal ends. Telomere-subtelomere fusion=junctions containing telomeric DNA presumably from one chromosome fused with another chromosome end with subtelomeric deletions. Complex fusion=fusion junctions containing non-telomeric DNA from interstitial chromosomal regions. FIG. 5C is a schematic drawing showing the detailed molecular structure of complex telomere fusion #14 from invasive tissue described in FIG. 5A. FIG. 5C is a schematic drawing showing the detailed molecular structure of complex telomere fusion #13 from invasive tissue described in FIG. 5A. SINE, short interspersed elements. LTR, long terminal repeat. Base pairs (bp), size of insertion.

FIGS. 6A-6D. Characterization of telomere fusion junctions from BJ E6/E7 cells, DCIS and invasive tumor tissue. FIG. 6A is a graph demonstrating the size of telomeric DNA at fusion junctions. FIG. 6B is a graph demonstrating the size of subtelomeric DNA deletion at fusion junctions. FIG. 6C is a graph demonstrating the distribution of microhomology by chance and observed microhomology at telomere fusions junctions from BJ E6/E7 (96 PDs), DCIS and IDC. By chance, the probability that a junction will have X nucleotides of microhomology by chance assuming an unbiased base composition. Black box, no shared nucleotides at junction; dark gray box, one shared nucleotide at junction; medium gray box, two shared nucleotides at junction; light gray box, three shared nucleotides at junction; open box, greater than or equal to four shared nucleotides at junction. FIG. 6D is a graph demonstrating the trinucleotide sequences of telomeric DNA termini at fusion junctions.

FIG. 7. The presence of telomere fusion in various cancers. TAR-fusion PCR assauy was performed to detect telomere fusion in each sample. The telomere fusion signals were detected by southern blot analysis described in Tanaka et. al., 2012, PNAS. 109:14098-14103.

FIG. 8 Three telomere fusion junctions in colon cancer were isolated. They are different from fusion junctions that were previously found in breast cancer, implying that chromosomes involved in telomere fusions in colon tumorigenesis could be unique. All fusion junctions contain telomeric DNA on one end fused to a subtelomeric region. A. The end of chromosome 7q fuses to another chromosome 7q subtelomere region. B. The end of chromosome Xp or Yp fuses to chromosome 15q subtelomeric region. C. The end of chromosome 4p fuses chromosome Xp or Yp subtelomeric region.

FIG. 9. Relationship between TP53 mutation and telomere fusion in colon cancer. This data shows that loss of the p53-dependent DNA damage response pathway may facilitate telomere fusions in human tissue, resulting in numerous genetic changes necessary for the emergence of nascent cancer cells. Preliminary observations show that even tumor surrounding tissue contains telomere fusions, presuming that telomere fusion emerges at an early stage of human carcinogenesis.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments above and the claims below. Reference should therefore be made to the embodiments above and claims below for interpreting the scope of the invention.

### DETAILED DESCRIPTION

The kits, methods and compositions now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements.

Likewise, many modifications and other embodiments of the kits, methods and compositions described herein will come to mind to one of skill in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

### DEFINITIONS

In describing and claiming the invention, the following terminology will be used in accordance with the definitions set forth below.

The term “isolated” as used herein refers to material that has been removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “purified” and like terms relate to the isolation of a molecule or compound in a form that is substantially free of contaminants normally associated with the molecule or compound in a native or natural environment.

As used herein, the term “primer” refers to an oligonucleotide, whether occurring naturally as in a purified restriction digest product, or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products.

As used herein, a “nucleic acid” sequence means a DNA or RNA sequence. The term encompasses sequences that include any of the known base analogues of DNA and RNA such as, but not limited to 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman (Smith and Waterman, Advances in Applied Mathematics, 2:482-489, 1981, Smith et al., Nucleic Acids Research 11:2205-2220, 1983), the homology alignment algorithm of Needleman and Wunsch (Needleman and Wunsch, Journal of Molecular Biology 48:443-453, 1970), the search for similarity method of Pearson and Lipman, and preferably by computerized implementations of these algorithms such as GAP, BESTFIT, FASTA, and TFASTA available as part of the GCG®. Wisconsin Package®. (Accelrys Inc., Burlington, Mass.). More particularly, computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990). For purposes of this invention “percent identity” may also be determined using BLASTX version 2.0 for translated nucleotide sequences and BLASTN version 2.0 for polynucleotide sequences.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished.

As used herein, the term “antibody” refers to a polyclonal or monoclonal antibody or a binding fragment thereof such as Fab, F(ab′)2 and Fv fragments.

The term “label” as used herein refers to any atom or molecule which can be used to provide a detectable (preferably quantifiable) “signal”, and which can be attached to a nucleic acid or protein. Labels or “detectable markers” may provide “signals” detectable by fluorescence, radioactivity, colorimetry, gravimetry, X-ray diffraction or absorption, magnetism, enzymatic activity, and the like.

As used herein, “biological sample” includes, but is not limited to, normal tissue samples, diseased tissue samples, biopsies, blood, saliva, feces, semen, tears, and urine. A biological sample can also be any other source of material obtained from a subject that contains cells, tissues, or fluid of interest. A sample can also be obtained from cell or tissue culture.

The term “neoplastic cells” as used herein refers to cells that result from abnormal new growth.

As used herein, the term “tumor” refers to an abnormal mass or population of cells that result from excessive cell division, whether malignant or benign, and all pre-cancerous and cancerous cells and tissues. A “tumor” is further defined as two or more neoplastic cells.

“Malignant cells/tumors” are distinguished from benign cells/tumors in that, in addition to uncontrolled cellular proliferation, they will invade surrounding tissues and may additionally metastasize. Cancer cells are cells that have undergone malignant transformation.

The term “neoplastic disease” as used herein refers to a condition characterized by uncontrolled, abnormal growth of cells. Neoplastic diseases include cancer.

### EMBODIMENTS

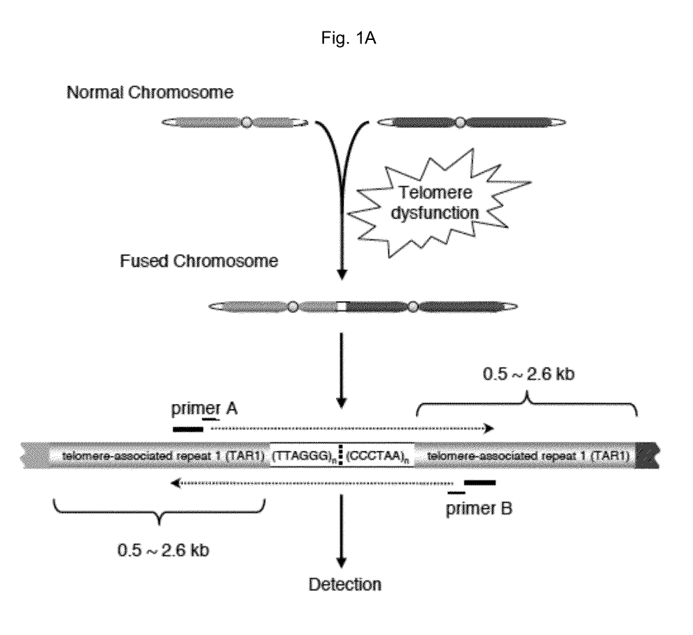

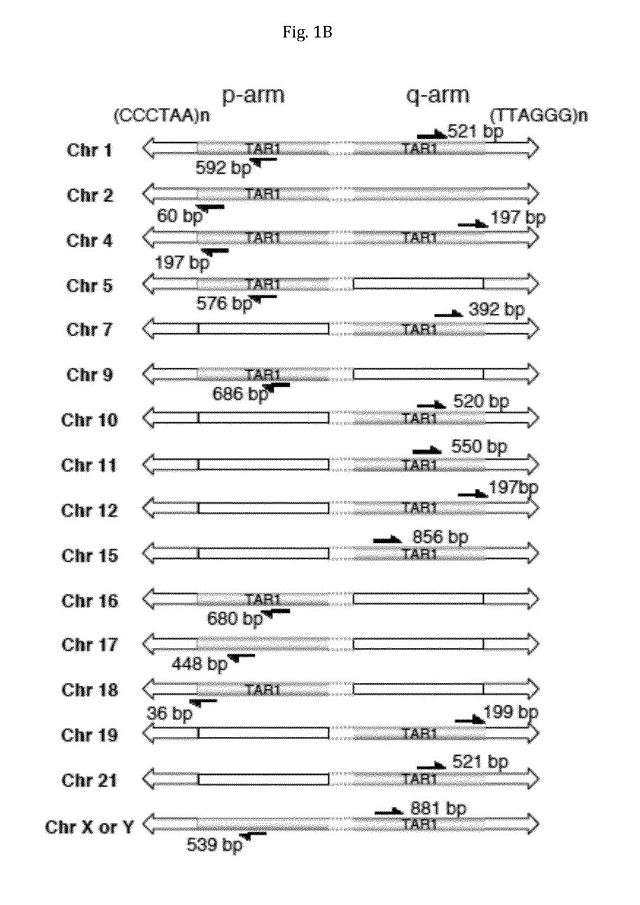

The present disclosure is based on the premise that telomere dysfunction is a driving force behind the genomic instability observed in early malignant lesions. The telomere dysfunction hypothesis states that telomere capping is disrupted in a small subset of normal precursor cells, leading to telomere fusions that ultimately cause genomic instability via breakage-fusion-bridge cycles (FIG. 1A). Consequently, loss of genomic integrity leads to mis-regulation of genes (including TERT—the catalytic component of telomerase) involved in growth control, ultimately resulting in tumorigenesis.

One aspect of the present disclosure is directed to compositions and methods for detecting signs of telomere dysfunction as diagnostic indicators of metastatic disease. More particularly, in accordance with one embodiment a method is provided for detecting the presence of telomere fusion products in the cells of a biological sample. Such telomere fusions have been associated with cancerous or pre-cancerous cells and can serve as early diagnostic markers of neoplastic disease.

In accordance with one embodiment purified or isolated nucleic acid sequences are provided that specifically bind to telomere associated repeats (TAR) and can be used as PCR primers to amplify telomere fusion products present in the cells of a biological sample. The presence of such telomere fusion products in the cells of a biological sample are diagnostic of cancer or pre-cancerous cells. In one embodiment the purified or isolated nucleic acid sequence are selected from the group consisting of:

SEQ ID NO: 1. GAG CTC CGT TTT CCT CTG CAG AGA CTT CG (4pq_TAR1);

SEQ ID NO: 2. GCC CCG GCG CAA CGG TGG CAT GCC TCT CTG GG (4pq_TAR2);

SEQ ID NO: 3. GCC CCG GCG CCA CGG TGG CAT GCC TCT CTC GG (4pq_TAR2*);

SEQ ID NO: 4. ATC TCT CCG CGT GAC TCT TGC CAC CGC CAC G (4pq_TAR3);

SEQ ID NO: 5. ATC TCT CCG CGT GAC TCT TGC CCC CGC CAC G (4pq_TAR3*);

SEQ ID NO: 6. GCA GCC GTG CAT CTC TCC GCG TGA CTC TTG (4pq_TAR5);

SEQ ID NO: 7. GTG CGC TTA TGC AGC TGT GCA CCT GCA GTA CG (2p_TAR3);

SEQ ID NO: 8. GAG CAC ACA CCC GGT GGA CAC CGT AAA GGC G (2p_TAR4);

SEQ ID NO: 9. GAG TTG CGT TCT CTG TAG CAC AGA CCC TAA CAG (5p_TAR5);

SEQ ID NO: 10. GCC CCT GTG AAT CCT GTA CAC CGA GATG (5p_TAR6);

SEQ ID NO: 11. GCA GCC CTC AAC AAT CAG GGT CAG AGT CCA G (18p_TAR1);

SEQ ID NO: 12. GAO TCG CAG AGC ATG GCG AGG GCG CAG (18p_TAR2);

SEQ ID NO: 13. GGC GCT GCG CCT TTG CGA AAG TGA AGC TGT G (18p_TAR3);

SEQ ID NO: 14. GCC TTT GCG AAA GTG AAG CTG TGT TCT CAT CAG CAG AG (18p_TAR4);

SEQ ID NO: 15. GCC ACC CGC CAA TAO ATT TGG AGC AAG AGG (11p_TAR1);

SEQ ID NO: 16. GCT TTG ACA CGA CTC GGG GCT TCA TTG ATG GTG (7qTAR1);

SEQ ID NO: 17. GCT TTG GCA CGA CTC GGG GCT TCA TTG ATG GTG (7q_TAR1*);

SEQ ID NO: 18. GCC TTT GCG AGG GCG GAG TTG CGT TCT CTT TAG (p-arm_TAR);

SEQ ID NO: 19. GCC GTG CGA CTT TGC TCC TGC AAC ACA CG (q-arm_TAR);

SEQ ID NO: 1 GAA CTG TGT TAO AGA CCA GGA GCT CAT GTA CAG GGC TG (Xp_ter); and

SEQ ID NO: 20. GGG ACA AGG CAA TTG TCA TGC AAT TAT CTA TGC AGG (17p_ter) or a nucleic acid sequence having at least 60%, 70%, 75%, 80%, 85%, 90%, or 95% sequence identity with any of the sequences after alignment to maximize matches, or any reverse complement of the sequences. In one embodiment the purified or isolated nucleic acid is DNA. In another embodiment the purified or isolated nucleic acid is labeled with a detectable marker.

In one embodiment a purified or isolated DNA sequence is provided that comprises a contiguous 20 nucleotide sequence that shares at least 80% sequence identity with a DNA sequence selected from the group consisting of SEQ ID NO: 1-20, or any reverse complement of the sequences. In a further embodiment the DNA sequences are labeled with a detectable marker. In one embodiment a composition is provided comprising 2, 3, 4, 5, 6, 7, 8, 9, 10, 11, 12, 13, 14, 15, 16, 17, 18, 19 or 20 of the telomere specific nucleic acid sequences disclosed herein.

In accordance with one embodiment, a method of detecting telomere fusions in the cells of a biological sample comprises the use of PCR to specifically amplify any telomeric fusion product present in a biological sample.

In one embodiment the biological sample represents nucleic acid sequences isolated from a patient's tissues, and more particularly in one embodiment the patient is a human. The tissue may comprise a blood sample or a solid tissue biopsy sample recovered from the patient. In one embodiment the biological sample represents human breast tissue. In other embodiments, the biological sample represents human ovarian, prostate, and colon tissue.

In accordance with one embodiment a method of detecting neoplastic or pre-cancerous cells in a patient is provided. The method can be used to detect any cancer and in one embodiment the method is used to detect breast cancer or a pre-cancerous tissues. In some embodiments the cancer is selected from: bladder cancer, brain cancer, breast cancer, colorectal cancer, cervical cancer, gastrointestinal cancer, genitourinary cancer, head and neck cancer, lung cancer, ovarian cancer, prostate cancer, renal cancer, skin cancer, and testicular cancer. Specific amplification of telomere fusions is accomplished through the use of a telomere specific PCR primer and appropriate reaction conditions. The method comprises contacting cellular DNA from tissues of the patient with one or more PCR primers to form a reaction substrate. The PCR primers are selected from the group consisting of SEQ ID NO: 1-20 or a nucleic acid sequence having at least 60%, 70%, 75%, 80%, 85%, 90%, or 95% sequence identity with any of the sequences after alignment to maximize matches, or any reverse complement of the sequences. A PCR amplification reaction is then conducted on the reaction substrate and the sample is then analyzed to determine if any amplification products were produced. Detection of amplified products indicates the presence of telomere fusions and such telomere fusions are associated with neoplastic or pre-cancerous cells.

In accordance with one embodiment the telomere-specific PCR primers are selected from the group consisting of SEQ ID NO: 1-20.

Absent the presence of a telomere fusion product in the biological sample, the PCR reaction will fail to produce an amplicon through the use of the telomere-specific PCR primer disclosed herein. As used herein each primer is named to designate the chromosome to which it binds as the first designated number followed by whether the primer binds to the q or p arm (or both) of the chromosome. For q-arm_TAR, the primer binds to multiple chromosomes including 1q, 10q, 19q, and 21q. For p-arm_TAR, the primer binds to multiple chromosomes including 1p, 9p, 16p, 15q, Xq and Yq.

In accordance with one embodiment, a method of detecting the presence of telomere fusions in a population of cells is provided comprising the step of contacting nucleic acid sequences of a biological sample with two or more primers selected from the group consisting of SEQ ID NO: 1-20 (or the reverse complement of such sequences), conducting PCR, and screening the sample to detect the presence of an amplified product. Detection of an amplicon produced by PCR indicates the presence of one or more telomere fusions in the biological sample. Furthermore, analysis of the sequence of the resultant amplicon can reveal the identity of the fused chromosomes. Analysis of the amplicon can be conducted by nucleic acid hybridization, sequence analysis, mass spectrometry or any other technique known to those skilled in the art.

In accordance with one embodiment, multiplex PCR is conducted on the biological sample. In one embodiment either the primer q-arm_TAR or p-arm_TAR is used as the primer for PCR on the DNA of the biological sample. In one embodiment any combination of 2 to 20 of the PCR primers selected from (4pq_TAR1), (4pq_TAR2), (4pq_TAR2*), (4pq_TAR3), (4pq_TAR3*), (4pq_TAR5), (2p_TAR2), (2p_TAR3), (2p_TAR4), (5p_TAR5), (5p_TAR6), (18p_TAR1), (18p_TAR2), (18p_TAR3), (18p_TAR4), (11p_TAR1), (7qTAR1), (7q_TAR1*), (p-arm_TAR), (q-arm_TAR), (Xp_ter), and (17p_ter) is used in the PCR. In a further embodiment, the multiplex PCR is conducted using 4, 6, 8, 10, 12, 14, 16, 18, 20 or more PCR primers wherein the 4, 6, 8, 10, 12, 14, 16, 18 or 20 PCR primers are selected from the group consisting of SEQ ID NO: 1-20 (or the reverse complement of such sequences). In accordance with one embodiment the biological sample contacted with the PCR primers comprises total DNA, or nuclear DNA, recovered (e.g., isolated or purified) from mammalian cells. In another embodiment the biological sample comprises thin sections of mammalian cells/tissues.

In accordance with one embodiment the cellular DNA of the biological sample is contacted with the telomere-specific PCR primer under conditions that allow the PCR primer to bind to its complementary strand on the target DNA. Suitable buffers and polymerase enzymes are then added to the reaction substrate, and a PCR amplification reaction is run using standard techniques known to those skilled in the art. In the absence of a telomere fusion, no DNA amplification will occur. After completion of the PCR amplification reaction, the sample is screened for the presence of an amplified product. Detection of the amplification product can be conducted using any of the known techniques used to detect the presence of nucleic acid sequences. In accordance with one embodiment the PCR primer is labeled with a detectable marker, and the production of a PCR amplicon can be detected based on the detection of a labeled nucleic acid amplified product that is larger in size than the original PCR primer. Alternatively, in one embodiment the PCR primer is not labeled and the amplification products are detected through the use of DNA intercalating agents (such as ethidium bromide), or other DNA binding entities that are labeled or produce a signal upon binding to DNA. An additional example includes the use of standard Southern blotting techniques known to those skilled in the art to detect the amplified product. Detection of an amplified product (i.e. a DNA segment greater in size then the original primer) indicates the presence of telomere fusions in the original cells of the biological sample. In accordance with one embodiment the amplified sequences can be cloned and sequenced, or analyzed using other techniques known to those skilled in the art, to provide further information regarding the detected telomere fusions.

The tissue selected for analysis can be any mammalian tissue and in one embodiment any human tissue. In one embodiment, the tissue represents a biopsy sample recovered from a human and in one embodiment the sample is human female breast tissue. In another embodiment the sample is human ovarian, prostate or colon tissue. In one embodiment, nuclei acid sequences are first isolated from the cells of the biological sample. More particularly, in one embodiment DNA, including total DNA or in one embodiment, genomic DNA is isolated from the cells. In another embodiment, the tissue or cells are collected from a biological sample. This isolated DNA is then contacted with the telomere specific PCR primer and the PCR amplification reaction is conducted. In an alternative embodiment the PCR reaction can be conducted in situ on thin slices of the biological tissue sample. In situ reactions offer the advantage of demonstrating the extent of telomere fusions in the cells of the tissue sample and may provide prognostic information as well as help define treatment strategies.

In another embodiment, a kit is provided for conducting the telomere-specific PCR amplification reactions of the present disclosure. Such a kit can be used to detect and analyze telomere fusions present in a biological sample. In accordance with one embodiment, the kit comprises PCR primers that specifically bind to and amplify telomere fusion sequences. The kit can be further provided with instructional materials, additional reagents and disposable labware for conducting PCR amplifications.

In accordance with one embodiment, the telomere-specific PCR primers of the kit comprise two or more PCR primers selected from the group consisting of SEQ ID NO: 1-20, or any of their reverse complement sequences.

In accordance with one embodiment, the kit comprises at least 4 and up to 20 PCR primers selected from the group consisting of SEQ ID NO: 1-20, or any of their reverse complement sequences.

In one embodiment, the kit comprises 4, 6, 8, 10, 12, 14, 16, 18 or 20 PCR primers selected from the group consisting of SEQ ID NO: 1-20, or any of their reverse complement sequences.

In one embodiment, the kit comprises 8 or more PCR primers selected from the group consisting of SEQ ID NO: 1-20, or any of their reverse complement sequences.

In one embodiment, the kit comprises the PCR primers of SEQ ID NO: 1-20, or any reverse complementary sequence of such sequences.

In one embodiment, the kit further comprises reagents for conducting PCR, including, for example, suitable buffers and/or enzymes. In one embodiment, the kit comprises a nucleic acid polymerase including, for example, a thermostable polymerase. In one embodiment, the kit is provided with thermostable polymerase such as the Taq polymerase, for example. In another embodiment, the PCR primer provided with the kit is labeled, or reagents are provided for labeling the PCR primer or detecting the amplification product of the reaction. The detection reagents include, for example, DNA binding dyes and labeled probes that bind to telomere specific sequences. The nucleic acids and other reagents can be packaged in a variety of containers, e.g., vials, tubes, bottles, and the like. Other reagents can be included in separate containers and provided with the kit; e.g., positive control samples, negative control samples, buffers, etc.

In accordance with one embodiment, the kits of the present disclosure are used in a method of detecting telomere fusions in a biological sample. The method comprising contacting cellular DNA from the biological sample with one or more (e.g., 2, 4, 8, 10, 12, 14, 16, 18 or 20) PCR primers selected from the group consisting of SEQ ID NO: 1-20, to form a reaction substrate, conducting a PCR amplification reaction on the reaction substrate, and detecting the presence of amplified products, wherein the detection of an amplified product indicates the presence of telomere fusions. In one embodiment, the method is conduction on nucleic acid sequences of a biological sample recovered from a human patient. In one further embodiment, the PCR is conducted on nucleic acid sequence in situ. In an alternative embodiment, PCR is conducted on nucleic acid sequences isolated or purified from the biological sample. In one embodiment, the biological sample comprises human breast, ovarian, prostate and colon tissue and in a further embodiment the PCR is conducted on DNA sequences purified from human tissues, including for example female breast tissue.

### Example 1

### Use of Telomere Fusions as Diagnostic Markers of Cancer

Telomere dysfunction is one of the key driving forces behind the genomic instability observed in early breast lesions. As proposed by applicants, telomere capping is believed to be disrupted in a small subset of normal breast epithelial cells. This loss of telomere function then results in telomere fusions, causing genomic instability via breakage-fusion-bridge cycles during subsequent cell cycles. Telomere dysfunction is likely indicated by alterations in telomere-associated protein levels, disruption of critical telomere-associated protein-protein interactions, deregulation of telomere-associated protein modification (e.g., phosphorylation), loss of a critical tissue-specific telomeric DNA length, and by the accumulation of telomere fusions. Consequently, loss of genomic integrity leads to misregulation of genes involved in growth control (including hTERT—the catalytic component of telomerase), ultimately resulting in tumorigenesis.

Several recent reports support the theory that defects in telomere maintenance initiate genomic instability eventually resulting in the development of breast cancer and other cancers. However, the extent of telomere dysfunction in human breast cancer (and other cancers) has not been directly determined due to present methodological limitations in detecting telomere dysfunction in tissue. Telomere length can be readily determined in tissue, however, telomere shortening does not necessarily indicate loss of telomere function, and telomere dysfunction can occur without telomeric DNA shortening. Accordingly, the present disclosure provides reagents and methods for examining loss of telomere function during breast tumorigenesis.

To overcome current methodological limitations and to directly determining the extent of telomere dysfunction during breast tumorigenesis, assays were developed that detect telomere fusion in cell lines and tissue.

### Example 2

### A Multiplex PCR-Based Assay to Detect Chromosome End-to-End Fusions

Materials and Methods

Tissue collection. Frozen normal (20-65 yr; mean 29.5±13 yr) and tumor breast tissue (DCIS, 30-80 yr, mean=55±12 yr and IDC, 31-86 yr, mean=47.6±13.3 yr) samples were collected from the Indiana University Simon Cancer Center Tissue Bank and the Susan G. Komen Tissue Bank at the Indiana University Simon Cancer Center using an Indiana University School of Medicine IRB approved protocol. Immediately following surgical removal, the tissue specimen was placed in an iced sterile container and transferred to pathology for diagnosis and processing. Each aliquot of deidentified tumor tissue was assigned a unique barcode for identification. Each tumor tissue aliquot obtained contained approximately 100 to 250 mg of breast tumor tissue. All tumor tissues specimens were obtained by excisional biopsy and were reviewed histologically to confirm the absence or grade of breast carcinoma by a group of pathologists at the Indiana University Simon Cancer Center. The tumor cell content of tumor tissues used in this study ranged from 50 to 90%.

Cell Culture

Foreskin fibroblasts BJ cells were obtained from ATCC. Cells were maintained in advanced DMEM medium (Life Technologies) supplemented with L-glutamine, penicillin-streptomycin, 5% FBS at 37° C. in 5% CO₂. BJ ells (60 population doublings) were infected with a pLXSN retrovirus expressing HPV16 E6 and E7 proteins (15) and selected on G418 (400 μg/ml) after 3 weeks of continuous drug treatment.

TAR Fusion PCR

Genomic DNA from cultured cells was isolated by a salt precipitation method previously described (Miller et al., (1988) Nucleic Acids Res 16:1215-1215.). Genomic DNA was extracted from breast tissue using the Agencourt DNAdvance Kit (Beckman Coulter Inc.) with ˜1.5 μg of genomic DNA obtained from each ˜20 mg of breast tissue sample as determined by a Nanodrop 2000 spectrophotometer (Thermo Fisher Scientific Inc.). TAR Fusion PCR conditions outlined below were the same for both cultured cell and breast tissue DNA. Two-step touchdown PCRs were performed in a 20 ul reaction mixture using 50 ng of DNA, multiple primers, 10% 7-deaza-dGTP (Roche Diagnostics), and Advantage GC Genomic LA Polymerase Mix (Clontech Laboratories, Inc.). The cycling conditions were initially denatured at 94° C. for 3 min followed by 10 cycles of 94° C. for 30 sec, and 72° C. (−0.4° C. each cycle to a ‘touchdown’ at 68° C.) for 5 min, and then 20 cycles of 94° C. for 30 sec, 68° C. for 5 min. Primer mix A contained eight primers that anneal within TAR1 regions (with the exception of Xp and 17p): 1p, 2p, 5p, 7q, 9p, 11q, 12q, 15q, 16p, 17p, 18p, Xp, and Xq (FIG. 1B). Primer mix B contained eight primers that anneal within TAR1 regions (with the exception of Xp and 17p): 1q, 2p, 4p, 4q, 5p, 7q, 10q, 12q, 17p, 18p, 19q, 21q, and Xp (FIG. 1B). Table 1 provides the sequences corresponding to the primers used in primer mix A and B. TAR Fusion PCR products were then resolved on a 0.8% agarose gel and Southern analysis was performed using a ³²P-labeled [TTAGGG]₄ probe (Huda et al., (2007) Aging Cell 6:709-713).

To calculate the total number of theoretical telomere fusion combinations, and the percentage of telomere fusions detectable by TAR Fusion PCR, we used the general equation: C(n, 2)+n. n equals the number of unique chromosomal ends. The equation can be simplified to n (n+1)/2. Therefore, in human somatic cells with 46 unique chromosomal ends, the total number of theoretical telomere fusion combinations is: 46×(46+1)/2=1081. With the current TAR-fusion PCR primer mixes A and B, each primer mix covers 13 chromosomal arms (n=13 for each primer mix). Therefore, the number of detectable fusion combinations is 13×14/2=91 for each mix. The two primer mixes share 7 primers. Therefore, 7×8/2=28 combinations are detected by

TABLE 1
TAR Fusion PCR primer sequences

| Primer | Sequence | Tel region* | Primer mix |
|---|---|---|---|
| 2p_TAR | 5′-GCA GCA CTG AAT ACT CAA AGT CAG ACA CGA GTT AGG-3′ | 2p | A, B |
| 4pq_TAR | 5′-GAG CTC CGT TTT CCT CTG CAG AGA CTT CG-3′ | 4p, 4q | B |
| 5p_TAR | 5′-GAG TTG CGT TCT CTG TAG CAC AGA CCC TAA CAG-3′ | 5p | A, B |
| 7q_TAR | 5′-GCT TTG ACA CGA CTC GGG GCT TCA TTG ATG GTG-3′ | 7q, 12q | A, B |
| 11q_TAR | 5′-GCC ACC CGC CAA TAC ATT TGG AGC AAG AGG-3′ | 11q | A |
| 17p_ter | 5′-GGG ACA AGG CAA TTG TCA TGC AAT TAT CTA TGC AGG-3′ | 17p | A, B |
| 18p_TAR | 5′-GCA GCC CTC AAC AAT CAG GGT CAG AGT CCA G-3′ | 18p | A, B |
| p-arm_TAR | 5′-GCC GTG CGA CTT TGC TCC TGC AAC ACA CG-3′ | 1p, 9p, 16p, | A |
| | | 15q, Xq | |
| q-arm_TAR | 5′-GCC TTT GCG AGG GCG GAG TTG CGT TCT CTT TAG-3′ | 1q, 10q, | B |
| | | 19q, 21q | |
| Xp_ter | 5′-GAA CTG TGT TAC AGA CCA GGA GCT CAT GTA CAG GGC TG-3′ | Xp | A, B |

*Primer anneals at single or multiple chromosome end regions. See FIG. 1B for details.

both mixes. As a result, the total number of detectable fusion combinations is 91+91−28=154, and the coverage rate is 154/1081×100%=14.2%.

Telomere Length and Telomerase Activity

Genomic DNA was isolated and telomere length was measured by in-gel hybridization as described previously (Huda et al., (2007) Aging Cell 6:709-713). Mean telomere lengths were calculated using the Excel® spreadsheet program TELORUN© as described (Huda et al., (2007) Aging Cell 6:709-713). Telomerase activity was measured by the telomeric amplification protocol (TRAP) using the TRAPeze telomerase detection kit (Millipore). Cells were lysed with ice-cold 1×CHAPS lysis buffer (10 mM Tris-HCl, pH 7.5, 1 mm MgCl₂, 1 mm EGTA, 0.1 mm benzamidine, 5 mm β-mercaptoethanol, 0.5% CHAPS, and 10% glycerol) containing RNase inhibitor at a final concentration of 100-200 U/mL. A reaction mixture of 50 uL containing 0.5 μg of protein extract, 10 μL of 5 XTRAP reaction mix (Tris buffer, primers, dNTPs and oligomer mix for amplification of 36-bp internal control band), and 2 units of Tag DNA polymerase was incubated for 30 minutes at 30° C. and subsequently subjected to denaturation at 95° C. for 5 min. PCR cycles were 95° C. for 30 seconds, 52° C. for 30 seconds and 72° C. for 30 seconds for 32 cycles. For direct visualization of the TRAP ladder all the PCR products were electrophoresed on a 12.5% nondenaturing polyacrylamide gel and stained with SYBR Green.

Fluorescence In Situ Hybridization (FISH)

Cultured cells were treated with 0.1 μM colcemid (Roche) for four hours and fixed in 3:1 (v/v) methanol-acetic acid. Metaphase chromosomes were prepared using standard methods (Gardner et al., (2011) Breast Cancer Research 13:R53). A pancentromere probe was prepared by the purification of a PCR product amplified with human genomic DNA (Roche) as a template and using the following degenerate alpha satellite primer pairs: 5′-ACA GAA GCA TTC TCA GAA-3′ and 5′-TTC TGA GAA TGC TTC TGT-3′ as previously reported (Ikeno M, et al. (1998) Nat Biotechnol 16:431-439). The pancentromere probe was labeled with spectrum-red dUTP fluorophore using nick translation (Abbot Laboratories) and hybridized to metaphase spreads. After hybridization, slides were washed in (50% Formamide/2×SSC) at 42° followed by one wash in 2×SSC at 37° C. Slides were mounted with DAPI (Vectashield; Vector Laboratories). Fluorescence images were captured using a SPOT RT camera mounted on a Leica CTR 5000 fluorescence microscope and processed using Adobe Photoshop. 20 metaphases were analyzed for each sample.

Cloning and Sequencing of Telomere Fusion Junction

DNA samples were purified from either PCR reactions or from gel-purified DNA fragments using the GENECLEAN Turbo kit (MP Biomedicals). Cloning and transformation were carried out with a TOPO XL PCR Cloning kit (Life Technologies). Clones containing the TTAGGG repeat sequences were identified by colony hybridization with radiolabeled [TTAGGG]₄ probe. Positive clones were isolated and fusion junctions were sequenced. Sequence alignments were performed using the UCSC Genome Bioinformatics database.

Results

A multiplex PCR-based assay was devised to detect and analyze chromosome end-to-end fusions from solid tumors, whose as the present disclosure shows in human breast tumor tissue (FIG. 1A). To amplify individual telomere fusion junctions, a mixture of primer sets was used that anneal within telomere associated repeat 1 (TAR1) distal chromosomal regions (FIG. 1B). TAR1 regions are present within ˜2 kb of telomere variant repeat regions at chromosome ends and contain portions of sequence homology with other TAR1 regions. Two multiplex primer sets were used to increase the coverage of potential chromosome fusion combinations and to reduce the total number of required PCR reactions. Two primers used in this assay anneal within distal regions of Xp and 17p are not within known TAR1 regions (FIG. 1B). Assuming that TAR1 region are essentially identical on homologous chromosome ends, it was calculated that the multiplex PCR primer sets used in this study cover ˜14% of the theoretically possible chromosome end-to-end fusion combinations [number of possible telomere fusion combinations=n(n+1)/2; n=number of chromosome ends].

Initially, the TAR Fusion PCR assay was developed using the well-characterized BJ foreskin fibroblast expressing HPV16 E6/E7 (BJ E6/E7) telomere crisis model, which accumulates telomere fusions with increasing population doublings (FIG. 2A, B). It was determined empirically that specific primer sequence sets and PCR conditions were important for specificity and sensitivity parameters to eliminate background signals from the vast majority of normal human genomic DNA. As a negative control for telomere fusions, primary BJ foreskin fibroblast cells were used that have comparatively few cells exhibiting low numbers of telomere fusions (FIG. 2A, B). As previously described, a decrease in mean telomere lengths was observed with these cell populations upon increasing cellular passage: BJ (50 PD; 7.6 kb), BJ E6/E7 (87 PD; 5.9 kb) and BJ E6/E7 (96 PD; 5.2 kb) (FIG. 2C) Zou et al., (2009) Mol Cell Biol 29:2390-2397. TAR Fusion PCR followed by Southern blot analysis using a ³²P-labeled [TTAGGG]₄ probe revealed an increasing number of TAR Fusion PCR products with increasing BJ E6/E7 population doublings (FIG. 2D, E). TAR Fusion PCR products were then cloned to isolate specific fusion junctions and sequenced to determine the composition of these products. Most TAR Fusion PCR products were found to contain shortened telomeric DNA ˜60 to 700 bp in length adjacent to subtelomeric DNA at the fusion junctions (78.6%) (FIG. 3A, B; FIGS. 6A and B). Another class of less frequent telomere fusion junctions contained shortened telomeric DNA at the fusion junction (˜40 to 360 bp) presumably from both fused chromosome ends (FIG. 3A, B). TAR Fusion PCR was compared with a previously presented method for chromosome end-to-end fusion junction analysis finding similar results within fusions found in this crisis cell model (Letsolo et al., (2010) Nucleic Acids Res 38:1841-1852). The only major difference between these two PCR-based methods was that using TAR Fusion PCR evidence of telomere-to-telomere fusion junctions was found (FIGS. 3A and B). Therefore, it was concluded that TAR Fusion PCR amplifies actual telomere fusions. Our conclusion is supported by the correlation between increasing accumulation of PCR products with increasing frequency of telomere fusions as determined by metaphase analysis and confirmation of chromosome fusion junctions by sequence analysis.

Next TAR Fusion PCR was used to determine whether human normal and tumor breast tissue contained telomere fusions. It was found that ˜40% of both DCIS (n=25) and IDC (n=23) contained telomere fusions whereas no telomere fusions were found in normal controls (n=23) (FIGS. 4A and B). In addition, it was determine that the frequency of fusions within individual positive tumor tissue was similar regardless of tumor stage (FIG. 4C). Therefore, the percentage of breast tumors positive for telomere fusion and the frequency of fusion accumulation within individual tumor tissues remained constant from the DCIS to IDC stage of breast carcinoma. Sequence analysis revealed that the majority of fusion junctions from breast tumor tissue contained shortened telomeric DNA tracks of 197 to ˜700 bp in length from one chromosome fused to subtelomeric DNA regions from another distal chromosome end (90.9% for DCIS and 78.6% for IDC tumor tissue; FIGS. 5A and B; FIGS. 6A and B). Additionally, it was found that evidence of telomeric DNA to telomeric DNA fusion junctions containing telomeric DNA likely from both chromosomes involved in the fusion (FIGS. 5A and B). Interestingly, IDC also contained, at a lower frequency, complex fusion junctions with short regions of non-telomeric interstitial genomic DNA regions (211 to 447 bp) (FIGS. 5C and D). These short regions found within fusion junctions were composed of LTR and non-LTR Short INterspersed Elements (SINE) retrotransposon elements (FIGS. 5C and D). Analysis of microhomology revealed that fusions from both the BJ E6/E7 telomere crises model and invasive breast tumor tissue contained significant levels of microhomology at fusion junctions (p<0.01; FIG. 6C). However, fusion junctions within DCIS breast tumor tissue did not contain significant levels of microhomology (p=0.15; FIG. 6C). Additionally, junction sequence analysis revealed that the majority of telomeric DNA found at fusion junctions ended in two or three G residues (FIG. 6D). These results indicate that telomere fusions are present in human breast tumor tissue and fusion junction sequence analysis has provided important clues into potential mechanisms of chromosome end-to-end fusion events during human breast carcinogenesis.

### Example 3

TAR-fusion PCR assay was performed to detect telomere fusion in different types of cancer (FIG. 7). DNA isolated from frozen tissue samples obtained from the Indian University Cancer Center Tissue Bank were used to determine telomeric fusions. In colon cancer, in both invasive and non-invasive colon cancer had increases in the presence of telomere fusions, with non-invasive colon cancer having more fusions. Ovarian cancer had increased telomere fusions, while a benign ovarian tumor did not show any fusions. For prostate cancer, there was a large amount of telomere fusions detected, nearly double that of the prostate pre-cancer lesion. In breast cancer, both non-invasive and invasive breast cancer had telomere fusions, with non-invasive cancer having slightly more fusions.

In colon cancer, three fusion junctions were found that different from what was previously found in breast cancer (FIG. 8). These included the end of chromosome 7q fusing to another chromosome 7q subtelomeric region, the end of chromosome Xp or Yp fuses to chromosome 15 q subtelomeric region, and the end of chromosome 4p fuses to chromosome Xp or Yp subtelomeric region. It was also observed that in colon cancer, the loss of p53 dependent DNA damage response pathway may facilitate telomere fusions in human tissue, resulting in numerous genetic changes necessary for emergence of nascent cancer cells (FIG. 9). Preliminary observations have shown that even tumor surrounding tissue contain telomere fusions, showing that telomere fusion emerges at an early stage of human carcinogenesis, and could be useful to detect early stage cancer or increased risk of developing cancer.

It was examined whether telomere dysfunction occurs in human breast cancer using a novel TAR Fusion PCR technique. The study revealed that telomere fusions accumulate in a relatively early stage of breast carcinoma (DCIS) and remain at similar levels in the more advanced stage of invasive ductal carcinoma as detected by TAR Fusion PCR and confirmed by fusion junction sequence analysis. Telomere dysfunction may be limited during carcinogenesis by telomerase reactivation and other factors to prevent total genomic catastrophe, which would likely result in the loss of essential cellular functions required to maintain tumor cell viability. Our finding that telomere fusions appear in DCIS correlates with previous findings that telomere shortening, telomerase activation and other genomic aberrations are detected in DCIS. These results show that telomere dysfunction is a prevalent event during breast carcinogenesis and that telomere end-to-end fusions may be causal in driving genomic instability via breakage-fusion-bridge cycles.

The current TAR Fusion PCR method utilizes only partial primer coverage of chromosome ends due to the absence of sequence information for the majority of chromosome termini that is presently lacking in genomic sequence databases (FIG. 1B, open rectangles). It is estimated that our current method detects about 14% of the theoretically possible telomere fusion combinations. Despite this current limitation, we determined that ˜40% of breast tumor tissue (both DCIS and IDC) tested contained detectable telomere fusions. Therefore, even though our current method only covers ˜14% of the possible chromosomal end fusion combinations, telomere dysfunction appears to be a prevalent genetic aberration event in breast tumorigenesis (approximately 40% of breast tumor tissue, both DCIS and IDC, tested contained telomere fusions. Using the presence of telomere fusions for early detection of breast cancer has potentially powerful advantages as a cancer biomarker because chromosome fusions are absent in normal tissue ((FIG. 4). Additionally, detection of telomere fusions could be used as an adjunct modality to support or refute a diagnosis of breast cancer when histopathologic findings are equivocal. In addition, the high frequency of telomere fusions we detected also supports the potential of these fusions as a cancer biomarker in the range of one of the most commonly mutated genes in cancer, p53.

The sequence analysis of the present disclosure at fusion junctions has provided some insight into potential mechanisms responsible for chromosomal end-to-end fusions. It was found that the majority of telomere fusions contain relatively short regions of telomeric DNA (˜100 to 880 bp) from one or both chromosomal ends involve in the fusion event. The shortened tracks of telomeric DNA may indicate that telomeric DNA is relatively shortened prior to fusion. Additionally, the majority of telomeric DNA found at fusion junctions ended in two or three G residues. However, chromosome ends during or prior to end-to-end fusion may be processed by an active mechanism. Therefore, the static composition of fusion junctions does not necessarily provide information regarding the structure of the chromosome end prior to fusion events but may provide information regarding mechanistic pathways. Interestingly, it was found that significant levels of microhomology at fusion junctions in both the telomere crisis model cells and invasive breast tissue but not in DCIS tissue (FIG. 6C). A portion of fusion junctions present in the crisis model cells and invasive tissue may be the result of mechanisms distinct from those generally found in DCIS potentially through pathways involving DNA-PKcs-independent nonhomologous DNA end joining/microhomology-mediated end joining.

In addition to the above, it was found that small regions of LTR and non-LTR retrotransposon elements from internal chromosomal regions within fusion junctions within human breast invasive tissue (FIGS. 5C and D). These more complex fusion junctions may be the resultant product of extensive breakage-fusion-bridge cycles that may occur during more advanced stages of breast carcinoma. Interestingly, similar retrotransposon elements are present at Drosophila chromosome ends and have been reported to integrate at dysfunctional mammalian telomeres in a Chinese hamster ovary cell line. The presence of retrotransposon elements at spontaneous fusion junctions within human invasive breast tissue may indicate an evolutionary conserved pathway for potential retrotransposon integration events at human dysfunctional telomeres. The presence of non-telomeric DNA retrotransposon elements within fusion junctions may also give us important additional leads for genetic biomarkers associated with tumorigenesis and potential mechanisms for genomic instability during breast tumorigenesis involving telomere dysfunction and other genomic interstitial regions vulnerable to breakage or recombination events.

It was also observed that these chromosome fusions could be detected in other types of tumors such as colon, ovarian, and prostate cancer. In colon cancer, tissue surrounding the tumor showed chromosome fusion, showing that the TAR Fusion PCR could be able to detect pre cancerous cells.

In summary, these results show that the occurrence of telomere dysfunction may be an early and potentially highly frequent genetic aberration event in the development of breast cancer, in addition to other types of cancer. Since detection of telomere fusions using TAR Fusion PCR was possible in the early stages of breast and colon cancer, these results show that it is possible to develop a simple, accessible clinical test for very early breast, prostate, ovarian and colon cancer detection using this approach.

The invention claimed is:

1. A kit for screening biological samples for the presence telomere fusions, the kit comprising,
one or more PCR primers selected from the group consisting of SEQ ID NO: 1-20, or any reverse complement of the sequences.

2. The kit of claim 1 further comprising a reagent for conducting PCR reactions.

3. The kit of claim 2, wherein the reagent comprises a thermostable polymerase.

4. The kit of claim 1, wherein the kit comprises one or more PCR primers selected from the group consisting of SEQ ID NO: 1-20, or a nucleic acid sequence having at least 95% sequence identity with any of the sequences.

5. The kit of claim 1, wherein the kit comprises one or more PCR primers selected from the group consisting of SEQ ID NO: 1-20, or a nucleic acid sequence having at least 90% sequence identity with any of the sequences.

6. The kit of claim 1, wherein the kit comprises one or more PCR primers selected from the group consisting of SEQ ID NO: 1-20, or a nucleic acid sequence having at least 85% sequence identity with any of the sequences.

7. The kit of claim 1, wherein the kit comprises one or more PCR primers selected from the group consisting of SEQ ID NO: 1-20, or a nucleic acid sequence having at least 80% sequence identity with any of the sequences.

8. The kit of claim 1, wherein the kit comprises one or more PCR primers selected from the group consisting of SEQ ID NO: 1-20, or a nucleic acid sequence having at least 75% sequence identity with any of the sequences.

9. The kit of claim 1, wherein the kit comprises 4 or more PCR primers selected from the group consisting of SEQ ID NO: 1-20, or any reverse complement of the sequence.

10. The kit of claim 1, wherein the kit comprises PCR primers identified by SEQ ID NO: 1-20.

11. A method of detecting telomere fusions in a biological sample, the method comprising:
contacting cellular DNA from the biological sample with one or more PCR primers selected from the group consisting of SEQ ID NO: 1-20, or a nucleic acid sequence having at least 60%, 70%, 75%, 80%, 85%, 90%, or 95% sequence identity with any of the sequences, or any reverse complement of the sequences, to form a reaction substrate;
conducting a PCR amplification reaction on the reaction substrate; and
analyzing the reaction substrate after PCR to detect the presence of amplified products, wherein the detection of an amplified product indicates the presence of telomere fusions in the biological sample.

12. The method of claim 11, wherein the reaction substrate comprises 4 or more of the PCR primers.

13. The method of claim 11, wherein the biological sample is selected from the group consisting of human breast, colon, ovarian, and prostate tissue.

14. The method of claim 11, wherein the PCR amplification reaction is conducted on DNA isolated from the cells of a patient.

15. The method of claim 11, wherein the PCR amplification reaction is conducted in situ on sectioned tissue obtained from a patient.

16. The method of claim 11, wherein the detection of telomere fusion is indicative of neoplastic or pre-cancerous cells.

16. A purified nucleic acid sequence selected from the group consisting of SEQ ID NO: 1-20, or a nucleic acid sequence having at least 80% sequence identity with any of the sequences, or any reverse complement of the sequences.

17. The nucleic acid sequence of claim 11 wherein the nucleic acid is a DNA sequence selected from the group consisting of SEQ ID NO: 1-20, or any reverse complement of the sequences.

18. The nucleic acid of claim 11, wherein the nucleic acid sequence is labeled with a detectable marker.
